(19) United States
(12) Patent Application Publication
(10) Pub. No.: US 2025/0145955 A1
(43) Pub. Date: May 8, 2025

(54) KIT AND METHOD FOR EXTRACTING CELL MITOCHONDRIA IN VITRO

(71) Applicant: SHANGHAI SIXTH PEOPLE'S HOSPITAL, Shanghai (CN)

(72) Inventors: Junjie GAO, Shanghai (CN); Changqing ZHANG, Shanghai (CN); Yiyang MA, Shanghai (CN)

(21) Appl. No.: 18/813,529

(22) Filed: Aug. 23, 2024

(30) Foreign Application Priority Data
Nov. 2, 2023 (CN) 202311450225.2

(51) Int. Cl. C12N 5/077

(52) U.S. Cl. CPC C12N 5/0654

(57) ABSTRACT

The invention provides a kit and a method for extracting cell mitochondria in vitro. The kit comprises an extracting solution A, an extracting solution B, an extracting solution C and a storage solution, wherein the extracting solution A comprises EDTA (Ethylene Diamine Tetraacetic Acid), sodium lactate and sodium chloride, the extracting solution B is a heparin solution, the extracting solution C comprises mannitol, EDTA, sodium lactate and sodium chloride, and the storage solution comprises mannitol, EDTA, sodium lactate, succinic acid, sodium pyruvate and sodium chloride. The functions of mitochondria can be better maintained in vitro by utilizing the extracting solution and the storage solution, the interval time from mitochondria extraction to mitochondria application to the body of a patient is prolonged, and beneficial help is provided for clinical application of mitochondria transplantation.

### BACKGROUND OF THE INVENTION

### Field of the Invention

The present invention relates to the field of biotechnology, particular to a kit and method for extracting cell mitochondria in vitro.

### Description of the Related Art

Mitochondria have been considered therapeutic targets in medicine for the past three decades. There is a wealth of research on targeting mitochondrial defects, suggesting great potential. In recent years, the use of mitochondrial transplantation to repair tissue function has attracted great interest in the academic community. So far, the application of mitochondrial transplantation has been expanded to various disease models, including ischemia-reperfusion injury, neurodegenerative diseases, kidney injury, ARDS, etc.

In vitro mitochondrial extraction preparation is the first step in applying mitochondrial transplantation to clinical practice. The main steps of in vitro mitochondrial extraction preparation are divided into: 1) cell lysis to isolate mitochondrial components, and 2) mitochondrial purification. Among them, mitochondrial lysis methods mainly include: 1) mechanical lysis methods, including: grinding homogenization, that is, by using specific grinding instruments (such as Dounce and Potter-Elvehjem homogenizers) to pass cells and tissues through narrow areas, causing them to break up and release cellular components; ultrasonic lysis, i.e., lysis of cells by low-frequency ultrasound; 2) chemical lysis method, including the use of digitonin or heparin, destroys the structure of the outer cell membrane and causes it to release cellular components. The purification methods of mitochondria mainly include: 1) differential centrifugation, which involves procedure with at least two different centrifugation speeds: low-speed centrifugation is used to discard large organelles such as nuclei, while high-speed centrifugation is used to precipitate mitochondria; 2) density gradient centrifugation, i.e., with the help of a discontinuous density gradient medium (such as Ficoll), particles of the same density in the cell lysate will be centrifuged to the same density layer to purify the mitochondria; 3) magnetic bead sorting: a method that uses magnetic beads as affinity material to capture mitochondria by antibody binding.

The extracted mitochondria need to be stored in the corresponding mitochondrial storage solution, and the next step needs to be carried out as soon as possible to prevent mitochondrial damage.

For mitochondrial transplantation, functional mitochondria with intact membrane potential and ATP-producing capacity need to be isolated. However, there are delicate trade-offs between the purity, yield, and function of isolated mitochondria. Functionally intact mitochondria are susceptible to contamination by non-mitochondrial components, and the removal of these non-mitochondrial components (e.g., endoplasmic reticulum) may in turn impair mitochondrial function. For cell lysis, grinding homogenate is the earliest method applied to scientific research, but it requires high precision and specific grinding instruments, which is not conducive to direct clinical use, and the method and frequency of grinding will lead to the difference in the degree of cell lysis and mitochondrial viability. The process of ultrasound will generate additional heat, and the long-term process will affect the viability of mitochondria, so chemical lysis method is a better method for clinical extraction of mitochondria because it does not require additional instruments and the concentration is controllable. However, at present, only commercially available imported mitochondrial extraction kits are used for chemical lysis, and the composition of their extraction reagents has not been announced, and there are no effective domestic alternatives. In addition, it is unclear which components and at what concentrations the extracted mitochondria are most suitable for mitochondrial transplantation.

At present, the internal formulation components of commercially available mitochondrial extraction and storage solution kits have not been disclosed. According to the literature, the commonly used mitochondrial storage mainly includes the following components: osmotic pressure regulating solutions (such as mannitol, sorbitol, sucrose), ion buffers (such as Tris-Hcl, HEPES), and calcium ion chelators (such as EGTA, EDTA). However, mitochondria preserved with the current formulation will lose membrane potential and respiratory function in a relatively short period of time, and cannot be used in clinical practice in a short period of time due to their complex composition.

### SUMMARY OF THE INVENTION

In order to overcome the defects in the prior art, there is provided a kit and a method for extracting cell mitochondria in vitro.

In order to achieve the above purpose, the present invention adopts the following technical solution.

The first aspect of the invention is to provide a kit for extracting cell mitochondria in vitro, comprising: an extracting solution A, an extracting solution B, an extracting solution C and a storage solution;

wherein the extracting solution A comprises EDTA, sodium lactate and sodium chloride, the extracting solution B is a heparin solution, the extracting solution C comprises mannitol, EDTA, sodium lactate and sodium chloride, and the storage solution comprises mannitol, EDTA, sodium lactate, succinic acid, sodium pyruvate and sodium chloride.

Further, the extracting solution A is made up of EDTA, sodium lactate and sodium chloride, the extracting solution C is made up of mannitol, EDTA, sodium lactate and sodium chloride, and the storage solution is made up of mannitol, EDTA, sodium lactate, succinic acid, sodium pyruvate and sodium chloride.

Further, the extracting solution A, counted as 100 ml of 0.6% sodium chloride solution, is prepared by the following method: 60˜90 mg EDTA, 200˜450 mg sodium lactate were dissolved in 100 ml of 0.6% sodium chloride solution, and filtered to prepare the solution A; preferably, 87.6 mg EDTA and 310 mg sodium lactate were dissolved in 100 ml of 0.6% sodium chloride solution, and filtered to prepare the solution A.

Further, the mass-to-volume ratio of the heparin solution is 1˜10%, preferably 4%.

Further, the extracting solution C, as counted 100 ml of 0.9% sodium chloride solution, is prepared by the following method: 2˜6 g mannitol, 60˜90 mg EDTA, 200˜450 mg sodium lactate were dissolved in 100 ml of 0.9% sodium chloride solution, and filtered to prepare the solution C; preferably, 4.55 g mannitol, 87.6 mg EDTA, and 310 mg sodium lactate were dissolved in 100 ml of 0.9% sodium chloride solution, and filtered to prepare the solution C.

Further, the storage solution, counted as 100 ml of 0.9% sodium chloride solution, is prepared by the following method: 2˜6 g mannitol, 60˜90 mg EDTA, 200˜450 mg sodium lactate, 100˜150 mg succinic acid, 40˜60 mg sodium pyruvate were dissolved in 100 ml 0.9% sodium chloride solution, filtered to prepare the storage solution; preferably, 4.55 g mannitol, 87.6 mg EDTA, 310 mg sodium lactate, 135 mg succinic acid, and 55 mg sodium pyruvate were dissolved in 100 ml of 0.9% sodium chloride solution, filtered to prepare the storage solution.

The second aspect of the invention is to provide a method for extracting cell mitochondria in vitro using the kit mentioned above, comprises the steps of:
a) adding digested cells into the extracting solution A, vortex shaking the mixture, and placing the mixture on ice;
b) adding the extracting solution B, vortex shaking the mixture at intervals, and placing the mixture on ice;
c) adding the extracting solution C, mixing and centrifuging, taking the supernatant, centrifuging again, discarding the supernatant, precipitating the mixture as mitochondria;
d) resuspending the precipitate in the storage solution and placing the mixture on ice for later use.

Further, in step b), the mixture is placed on ice for 5 minutes, and is vortex oscillated for 5 seconds per minute.

Further, in step c), centrifuging is performed with 700 g, and lasts for 10 minutes.

Further, in step c), the second time of centrifuging is performed with 10000 g, and last for 15 minutes.

The present invention adopts the above technical solution, and compared with the prior art, has the following technical effects.

The functions of mitochondria can be better maintained in vitro by utilizing the extracting solution and the storage solution, the interval time from mitochondria extraction to mitochondria application to the body of a patient is prolonged, and beneficial help is provided for clinical application of mitochondria transplantation.

### BRIEF DESCRIPTION OF THE DRAWINGS

FIG. 1 shows the significant increase in the proliferation capacity of mitochondria-treated bEnd.3 cells in which Figure A and Figure B show the EdU staining results of mitochondria-treated bEnd.3 cells and the corresponding statistical plots, respectively.

FIG. 2 shows confocal microscopy imaging results after mitochondrial extraction.

### DETAILED DESCRIPTION

The present invention is described in detail and specifically through specific embodiments and drawings below to enable a better understanding of the present invention, but the following embodiments do not limit the scope of the present invention.

In the embodiment, if the method does not have special instructions, the conventional method is adopted, and if there is no special description, the conventional commercially available reagent or the reagent prepared according to the conventional method is used.

### Example 1

The example provides a kit for extracting cell mitochondria in vitro, comprising: an extracting solution A, an extracting solution B, an extracting solution C and a storage solution.

Wherein the extracting solution A was prepared by the following method: 87.6 mg EDTA and 310 mg sodium lactate were dissolved in 100 ml of 0.6% sodium chloride solution, and then filtered by a 0.22 μm filter to prepare solution A;

the extracting solution B was prepared by the following method: 200 mg of heparin was dissolved in 5 ml of deionized water, and then filtered by a 0.22 μm filter to prepare solution B;

the extract solution C was prepared by the following method: 4.55 g mannitol, 87.6 mg EDTA, and 310 mg sodium lactate were dissolved in 100 ml of 0.9% sodium chloride solution, and then filtered by a 0.22 μm filter to prepare solution C;

the storage solution was prepared by the following method: 4.55 g mannitol, 87.6 mg EDTA, 310 mg sodium lactate, 135 mg succinic acid, 55 mg sodium pyruvate were dissolved in 100 ml of 0.9% sodium chloride solution, and then filtered by a 0.22 μm filter to prepare the storage solution.

### Example 2

The example provides a method for extracting cell mitochondria in vitro using the kit as provided in Example 1, comprises the steps of:
a) adding digested cells into 10 ml of extracting solution A, vortex shaking the mixture for 10 seconds, and placing the mixture on ice for 2 minutes;
b) adding 1 ml of extracting solution B, placing the mixture on ice for 5 minutes, with vortex shaking of the mixture for 5 seconds per minute, and;
c) adding 10 ml of extracting solution C, inverting and mixing the mixture, and centrifuging with 700 gravities for 10 minutes, taking the supernatant to a new centrifuge tube, centrifuging the supernatant with 10000 gravities for 15 minutes, discarding the supernatant, precipitating the mixture as mitochondria;
d) resuspending the precipitate in the storage solution and placing the mixture on ice for later use.

### Example 3

In this example, bEnd.3 cells are stimulated with extracted osteocyte mitochondria, and the specific steps of experiment and results are as follows:

We cultured MLO-Y4 cells in vitro, added 10 ml of solution A after digestion, vortex shook for 10 seconds and placed on ice for 2 minutes. Subsequently, 1 ml of B solution was added, placed on ice for 5 minutes, and vortex shook for 5 seconds per minute. Then, 10 ml of C solution was added, the mixture was inverted and mixed, and centrifuged for 10 minutes with 700 gravities, the supernatant was taken and transferred to a new centrifuge tube, centrifuged for 15 minutes with 10000 gravities, the supernatant was discarded, and the mixture was precipitated as mitochondria. The extracted mitochondria were resuspended with mitochondrial storage solution and placed on ice for later use. The extracted mitochondria were transplanted into the mouse vascular endothelial cell line bEnd.3 at a donor/recipient cell ratio of 10:1 (i.e., mitochondria extracted from 10 donor cells were transplanted into 1 recipient cell) and their proliferation ability was detected by EdU staining.

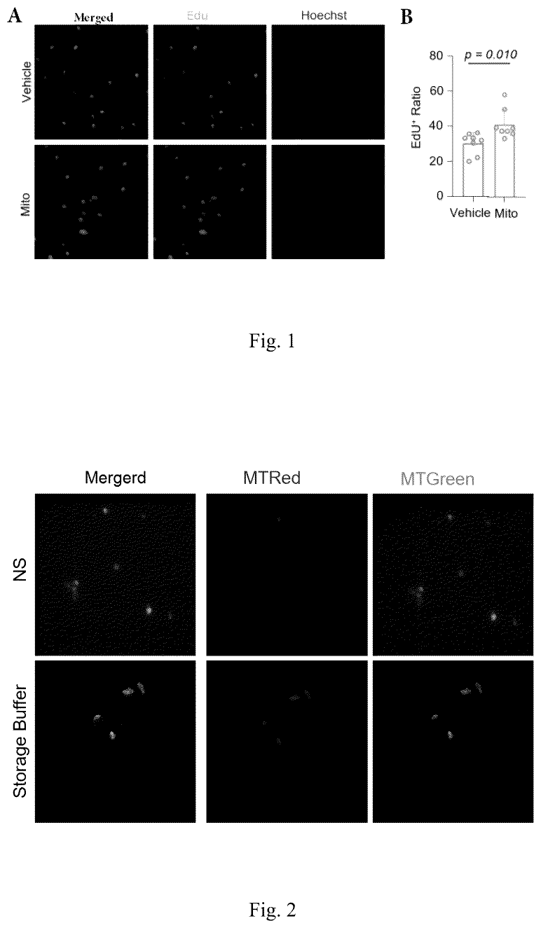

The result was shown in FIG. 1, the proportion of positive cells in mitochondria-treated bEnd.3 cells increased significantly, suggesting that the proliferative capacity of bEnd.3 was upregulated by the mitochondria extracted and stored by the method provided in Example 2.

### Example 4

The example verifies that the kit provided in embodiment 1 can make the extracted mitochondria maintain membrane potential and respiratory function for a long time, and the specific steps of the experiment and results are as follows:

20 ml of human whole blood was collected, centrifuged with 400 gravities for 10 minutes and the supernatant was taken, the supernatant was centrifuged with 1000 gravities for 10 minutes to obtain human platelets, 2 ml of A solution was added, vortex shook for 10 seconds and then placed on ice for 2 minutes. Subsequently, 0.2 ml of solution B was added, placed on ice for 5 minutes with vortex shaking for 5 seconds per 1 minute. Then, 2 ml of C solution was added, inverted and mixed well, centrifuged for 10 minutes with 700 gravities, the supernatant was taken and transferred to a new centrifuge tube, centrifuged for 15 minutes with 10000 gravities, the supernatant was discarded, and platelet-derived mitochondria were obtained. The extracted mitochondria were resuspended in normal saline and storage solution respectively, and placed on ice for 2 hours. Then, extracted mitochondria were co-stained with MitoTracker™ Green (Thermo M7514, a membrane potential-independent mitochondrial dye) and MitoTracker™ Red (Thermo M22425, a membrane potential-dependent mitochondrial dye), and photographed by confocal microscopy to observe mitochondrial membrane potential levels.

The mitochondrial staining results are shown in FIG. 2, and the MitoTracker Red staining of the mitochondria resuspended in the storage solution is darker, indicating that the mitochondrial membrane potential is well preserved.

The specific embodiments of the present invention are described in detail above, but they are only used as examples, and the present invention is not limited to the specific embodiments described above. For those skilled in the art, any equivalent modification and substitution of the present invention is also within the scope of the present invention. Therefore, equal transformations and modifications made without departing from the spirit and scope of the present invention shall be covered within the scope of the present invention.

What is claimed is:

1. A kit for extracting cell mitochondria in vitro, comprising: an extracting solution A, an extracting solution B, an extracting solution C and a storage solution;
wherein the extracting solution A comprises EDTA, sodium lactate and sodium chloride, the extracting solution B is a heparin solution, the extracting solution C comprises mannitol, EDTA, sodium lactate and sodium chloride, and the storage solution comprises mannitol, EDTA, sodium lactate, succinic acid, sodium pyruvate and sodium chloride.

2. The kit of claim 1, wherein the extracting solution A is made up of EDTA, sodium lactate and sodium chloride, the extracting solution C is made up of mannitol, EDTA, sodium lactate and sodium chloride, and the storage solution is made up of mannitol, EDTA, sodium lactate, succinic acid, sodium pyruvate and sodium chloride.

3. The kit of claim 2, wherein, the extracting solution A, counted as 100 ml of 0.6% sodium chloride solution, is prepared by the following method: 60˜90 mg EDTA, 200˜450 mg sodium lactate were dissolved in 100 ml of 0.6% sodium chloride solution, and filtered to prepare the solution A; preferably, 87.6 mg EDTA and 310 mg sodium lactate were dissolved in 100 ml of 0.6% sodium chloride solution, and filtered to prepare the solution A.

4. The kit of claim 2, wherein the mass-to-volume ratio of the heparin solution is 1˜10%, preferably 4%.

5. The kit of claim 2, wherein the extracting solution C, as counted 100 ml of 0.9% sodium chloride solution, is prepared by the following method: 2˜6 g mannitol, 60˜90 mg EDTA, 200˜450 mg sodium lactate were dissolved in 100 ml of 0.9% sodium chloride solution, and filtered to prepare the solution C; preferably, 4.55 g mannitol, 87.6 mg EDTA, and 310 mg sodium lactate were dissolved in 100 ml of 0.9% sodium chloride solution, and filtered to prepare the solution C.

6. The kit of claim 2, wherein the storage solution, counted as 100 ml of 0.9% sodium chloride solution, is prepared by the following method: 2˜6 g mannitol, 60˜90 mg EDTA, 200˜450 mg sodium lactate, 100˜150 mg succinic acid, 40˜60 mg sodium pyruvate were dissolved in 100 ml 0.9% sodium chloride solution, filtered to prepare the storage solution; preferably, 4.55 g mannitol, 87.6 mg EDTA, 310 mg sodium lactate, 135 mg succinic acid, and 55 mg sodium pyruvate were dissolved in 100 ml of 0.9% sodium chloride solution, filtered to prepare the storage solution.

7. A method for extracting cell mitochondria in vitro using the kit of claim 1, comprises the steps of:
a) adding digested cells into the extracting solution A, vortex shaking the mixture, and placing the mixture on ice;
b) adding the extracting solution B, vortex shaking the mixture at intervals, and placing the mixture on ice;
c) adding the extracting solution C, mixing and centrifuging, taking the supernatant, centrifuging again, discarding the supernatant, precipitating the mixture as mitochondria;
d) resuspending the precipitate in the storage solution and placing the mixture on ice for later use.

8. The method of claim 7, wherein in step b), the mixture is placed on ice for 5 minutes, and is vortex oscillated for 5 seconds per minute.

9. The method of claim 7, wherein in step c), centrifuging is performed with 700 g, and lasts for 10 minutes.

10. The method of claim 7, wherein in step c), the second time of centrifuging is performed with 10000 g, and last for 15 minutes.
